(19) United States
(12) Patent Application Publication
(10) Pub. No.: US 2013/0045959 A1
(43) Pub. Date: Feb. 21, 2013

(54) METHOD FOR LATE POST COITAL CONTRACEPTION USING ULIPRISTAL ACETATE

(71) Applicants: Andre Ulmann, Paris (FR); Henri Camille Mathe, Paris (FR); Erin Gainer, Paris (FR); Diana Blithe, Silver Spring, MD (US)

(72) Inventors: Andre Ulmann, Paris (FR); Henri Camille Mathe, Paris (FR); Erin Gainer, Paris (FR); Diana Blithe, Silver Spring, MD (US)

(21) Appl. No.: 13/575,918

(22) PCT Filed: Dec. 3, 2010
(86) PCT No.: PCT/EP2010/068863

§ 371 Date: Oct. 12, 2012

Related U.S. Application Data
(60) Provisional application No. 61/300,374, filed on Feb. 1, 2010.

(51) Int. Cl. A61K 31/57; A61P 15/18

(52) U.S. Cl. 514/179

(57) ABSTRACT

The invention provides a method for providing post coital contraception in a female subject, comprising providing the subject with a therapeutically effective amount of ulipristal acetate, between about 3 to about 5 days after unprotected intercourse.

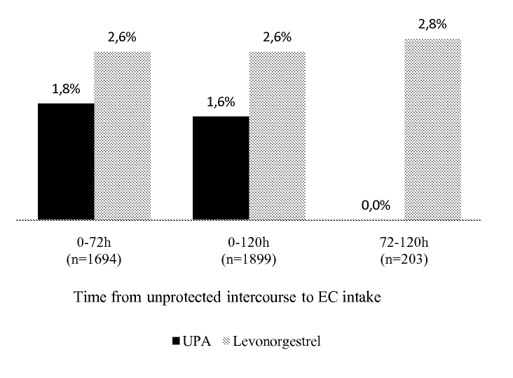

The present invention relates to a method for late post coital contraception, comprising administering ulipristal acetate to a female subject in need thereof.

### BACKGROUND TO THE INVENTION

Emergency contraception (EC) is a woman's second chance for primary prevention of pregnancy. A reproductive-age woman is a candidate for emergency contraception if she seeks care within 120 hours of unprotected intercourse (UPI), which is the window of pregnancy risk associated with a given act of intercourse based upon the estimated lifespan of sperm in the genital tract (Wilcox et al, 1995). Current hormonal methods of emergency contraception prevent at least half of expected pregnancies if taken within 72 hours of UPI (Von Hertzen et al, 1998).

Levonorgestrel at a total dose of 1.5 mg (taken in a single dose or two 0.75 mg doses 12 hours apart) is the current standard for hormonal emergency contraception and is licensed for use up to 72 hours after UPI. Clinical trials involving levonorgestrel used for emergency contraception more than 72 hours after intercourse do not conclusively establish efficacy rates because of insufficient sample size. Nevertheless, these studies reveal a trend towards markedly higher failure rates when levonorgestrel is taken 48 hours or more after unprotected intercourse (von Hertzen et al, 1998; Von Hertzen et al, 2002). This trend may be explained by levonorgestrel mode of action for emergency contraception. Levonorgestrel acts by interfering with the LH peak but does not appear to interfere with the ovulatory process when taken close to ovulation, a time when intercourse is most likely to lead to fertilization (Croxatto et al, 2004; Marions et al, 2004; Wilcox et al, 2004).

For a woman who presents for emergency contraception more than 72 hours after intercourse, the only currently available method proven to be highly effective is insertion of a copper contraceptive intra-uterine device (IUD). However, IUDs are not widely available in many countries and insertion can only be performed by a trained clinician. Furthermore, many women decline IUD insertion as a method of emergency contraception because the procedure is invasive, is relatively expensive and has a risk of complications including uterine perforation on insertion (Grimes et al, 2004). Additionally, many women seeking emergency contraception are not seeking a long acting contraceptive method. There is, therefore, a need for a new hormonal emergency contraceptive that can be used and is highly effective up to 120 hours after UPI.

Ulipristal acetate (also known as CDB-2914) is a selective progesterone receptor modulator that inhibits or delays ovulation in a dose-dependent fashion (Stratton et al, 2000). In a double-blind non-inferiority trial, ulipristal acetate was shown to be as efficacious as levonorgestrel for preventing pregnancy when used within 72 hours of UPI (Creinin et al, 2006). Moreover, study data suggest improved efficacy in preventing pregnancy from 48 to 72 hours when levonorgestrel efficacy markedly wanes.

### SUMMARY OF THE INVENTION

The invention provides ulipristal acetate for use in providing post coital contraception in a female subject between about 3 to about 5 days, or between about 72 to about 120 hours, after unprotected intercourse.

A subject of the invention is thus a method for providing post coital contraception in a female subject, comprising providing the subject with a therapeutically effective amount of ulipristal acetate, between about 3 to about 5 days, or between about 72 to about 120 hours, after unprotected intercourse.

It is further provided a kit comprising i) a dosage form comprising ulipristal acetate and ii) a printed matter stating that ulipristal acetate may be taken within 120 hours or 5 days after unprotected intercourse.

### LEGEND TO THE FIGURE

The attached FIGURE is a graph that shows pregnancy rates (ulipristal acetate vs levonorgestrel) according to time to emergency contraception.

### DETAILED DESCRIPTION OF THE INVENTION

Ulipristal acetate, formerly known as CDB-2914, designates within the context of this application 17α-acetoxy-11β-[4-N,N-dimethylamino-phenyl)-19-norpregna-4,9-diene-3,20-dione, represented by formula I:

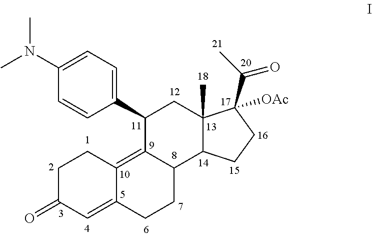 [SMILES: CC(=O)O[C@]1(C(C)=O)CCC2C3CCC4=CC(=O)CCC4=C3[C@@H](c3ccc(N(C)C)cc3)C[C@@]21C]

Ulipristal acetate, and methods for its preparation, are described e.g., in U.S. Pat. Nos. 4,954,490; 5,073,548, and 5,929,262, as well as in international patent applications WO2004/065405 and WO2004/078709, incorporated herein by reference.

Its main metabolite is monodemethylated CDB-2914 (CDB-3877A), that is 17α-acetoxy-11β-[4-N-methylamino-phenyl)-19-norpregna-4,9-diene-3,20-dione.

The subject, who may be also designated by the term “patient”, may be any woman in need of a post-coital contraception, preferably an emergency contraception.

Any woman of reproductive age may need post-coital or emergency contraception at some point to avoid an unintended pregnancy. It is meant to be used in situations of unprotected intercourse, such as:

when no contraceptive has been used;
when there is a contraceptive failure or incorrect use, including:
condom breakage, slippage, or incorrect use;
non-compliance with dosage regimen for combined oral contraceptive pills;
non-compliance with dosage regimen for progestogen-only pill (minipill);
more than two weeks late for a progestogen-only contraceptive injection (depot-medroxyprogesterone acetate or norethisterone enanthate);
more than seven days late for a combined estrogen-plus-progestogen monthly injection;
dislodgment, delay in placing, or early removal of a contraceptive hormonal skin patch or ring;
dislodgment, breakage, tearing, or early removal of a diaphragm or cervical cap;
failed coitus interruptus (e.g., ejaculation in vagina or on external genitalia);
failure of a spermicide tablet or film to melt before intercourse;
miscalculation of the periodic abstinence method or failure to abstain on fertile day of cycle;
IUD expulsion; or in cases of sexual assault when the woman was not protected by an effective contraceptive method.

Preferably post coital contraception is provided more than about 3 days, i.e. more than about 72 hours (exclusive, i.e. >72 hours) after unprotected intercourse. Preferably, post coital contraception is provided more than about 3, 4 and up to 5 or even 6 days after unprotected intercourse. Preferably, post coital contraception is provided more than about 75, 80, 90, or 96 hours after unprotected intercourse. Still more preferably, post coital contraception is provided up to 120 hours, preferably about 100, 110, 120 hours after unprotected intercourse.

In the present invention post coital contraception most preferably is an emergency contraception.

Ulipristal acetate may be administered by any convenient route, including oral, buccal, parenteral, transdermal, vaginal, uterine, rectal, etc.

For a brief review of present methods for drug delivery, see, Langer, Science 249:1527-1533 (1990), which is incorporated herein by reference. Methods for preparing administrable compounds are known or are apparent to those skilled in the art and are described in more detail in, for example, Remington's Pharmaceutical Science, 17th ed., Mack Publishing Company, Easton, Pa. (1985), which is incorporated herein by reference, and which is hereinafter referred to as “Remington.”

For solid compositions, conventional nontoxic solid carriers may be used which include, for example, pharmaceutical grades of mannitol, lactose, starch, magnesium stearate, sodium saccharine, talcum, cellulose, glucose, sucrose, magnesium, carbonate, and the like. For oral administration, a pharmaceutically acceptable nontoxic composition is formed by incorporating any of the normally employed excipients, such as those carriers previously listed.

Oral solid dosage forms preferentially are compressed tablets or capsules. Compressed tablets may contain any of the excipients described above which are diluents to increase the bulk of the ulipristal so that production of a compressed tablet of practical size is possible. Binders, which are agents which impart cohesive qualities to powdered materials are also necessary. Starch, gelatin, sugars such as lactose or dextrose, and natural and synthetic gums are used. Disintegrants are necessary in the tablets to facilitate break-up of the tablet. Disintegrants include starches, clays, celluloses, algins, gums and crosslinked polymers. Lastly small amounts of materials known as lubricants and glidants are included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Procedures for the production and manufacture of compressed tablets are well known by those skilled in the art (See Remington).

Capsules are solid dosage forms using preferentially either a hard or soft gelatin shell as a container for the mixture of ulipristal or a metabolite thereof and inert ingredients. Procedures for production and manufacture of hard gelatin and soft elastic capsules are well known in the art (See Remington).

Buccal forms or devices are also useful, such as those described in U.S. patent application 20050208129, herein incorporated by reference. U.S. patent application 20050208129 describes a prolonged release bioadhesive mucosal therapeutic system containing at least one active principle, with an active principle dissolution test of more than 70% over 8 hours and to a method for its preparation. Said bioadhesive therapeutic system comprises quantities of natural proteins representing at least 50% by weight of active principle and at least 20% by weight of said tablet, between 10% and 20% of a hydrophilic polymer, and compression excipients, and comprising between 4% and 10% of an alkali metal alkylsulphate to reinforce the local availability of active principle and between 0.1% and 1% of a monohydrate sugar.

For parenteral administration, fluid unit dosage forms are prepared utilizing the compounds and a sterile vehicle, water being preferred. Ulipristal acetate, depending on the vehicle and concentration used, can be either suspended or dissolved in the vehicle. In preparing solutions the compound can be dissolved in water for injection and filtered sterilized before filling into a suitable vial or ampoule and sealing. Advantageously, adjuvants such as a local anesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection is supplied to reconstitute the liquid prior to use. Parenteral suspensions can be prepared in substantially the same manner except that the compounds are suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of ulipristal acetate.

Additionally, a suppository can be employed to deliver ulipristal acetate. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate. These suppositories can weigh from about 1 to 2.5 g.

Transdermal delivery systems comprising a penetration enhancer and an occlusive backing are of use to deliver ulipristal acetate. Examples of penetration enhancers include dimethyl sulfoxide, dimethyl acetamide and dimethylformamide.

Systems comprising polymeric devices which slowly release or slowly erode and release within the body to provide continuous supplies of ulipristal acetate are also of use. Suitable delivery systems include subcutaneous devices or implants such as those routinely used to deliver norgestrienone or progestin 82323 and other medicaments.

Ulipristal acetate is preferably in form of an oral dosage, such as a tablet or a capsule, preferably a tablet.

In a preferred embodiment, it is provided as pharmaceutical tablet for oral administration, comprising ulipristal acetate in an amount of 3 to 18 wt %, together with the following excipients: a diluent in an amount of 60 to 95 wt %, a binding agent in an amount of 1 to 10 wt %, croscarmellose sodium in an amount of 1 to 10 wt %, and magnesium stearate in an amount of 0 to 5 wt %.

According to preferred embodiments, the composition, preferably in form of a tablet, comprises 10% wt ulipristal acetate and is designed to contain from about 5 to about 50 mg ulipristal acetate, preferably about 10, 20, or 30 mg.

The diluent may be selected from any pharmaceutically acceptable agent or combination of agents that increases the bulk quantity of ulipristal acetate so that production of a compressed tablet of practical size is possible. In a preferred embodiment, the diluent is selected from the group consisting of a monosaccharide, a disaccharide, a derivative polyol of a monosaccharide and hydrates thereof. The term ‘derivative polyol of a monosaccharide’ stands for a sugar alcohol such as mannitol, xylitol or sorbitol. Preferably the diluent is selected from the group consisting of lactose monohydrate and mannitol. In a most preferred embodiment, the diluent is lactose monohydrate is an amount of 65 to 92 wt %, more preferably 70-85 wt %.

The binding agent, or binder, may be selected from any pharmaceutically acceptable agent (or combination of agents) which imparts cohesive qualities to powdered materials. The binding agent may be selected from starch, gelatin, sugars such as cellulose derivatives, and natural and synthetic gums may be used. Advantageously, the binding agent of the tablet is selected from the group consisting of polymers. The binding agent may be a natural polymer material such as polysaccharide, or a synthetic polymer such as a plastic polymer. Preferably, the binding agent is hydroxypropyl methyl cellulose and/or povidone. In a preferred embodiment, the binding agent is or comprises povidone, preferably 1.5% to 8.5 wt % of povidone, even more preferably between 3-7 wt %, most preferably about 5 wt % povidone.

The tablets preferably comprise croscarmellose sodium. Croscarmellose sodium is a disintegrant, e.g., facilitates break-up of the tablet. Croscarmellose sodium may be used alone or in combination with other disintegrants, preferably alone. It is preferably present in an amount of 1 to 10 wt %, preferably 1.5 to 8.5 wt %, and more preferably 4.5 to 5.5 wt %, or even more preferably about 5 wt %.

In preferred embodiments, the tablets of the present invention contain magnesium stearate. While magnesium stearate may be used in combination with other lubricants, it is preferably used alone, in an amount comprised between 0.5 and 5 wt %.

Preferably, the tablet according to the present invention comprises lactose monohydrate as a diluent and povidone as a binding agent.

In a more specific embodiment, the tablet comprises: ulipristal acetate 5 to 15 wt %, lactose monohydrate 71 to 87 wt %, povidone 4.5 to 5.5 wt %, croscarmellose sodium 4.5 to 5.5 wt % and magnesium stearate 1 to 4 wt %, where the total percentage adds up to 100.

In an even more specific embodiment, the tablet comprises: ulipristal acetate 10%, lactose monohydrate 79 wt %, povidone 5 wt %, croscarmellose sodium 5 wt % and magnesium stearate 1 wt %.

Tablets may be prepared according to techniques known per se in the art. Suitable methods include direct compression (“dry blending”), dry granulation followed by compression, and wet granulation followed by drying and compression. Several methods include the use of compacting roller technology such as a chilsonator or drop roller, or molding, casting, or extrusion technologies. The tablet can be a coated tablet or an uncoated tablet.

In the preparation of the tablets, commercial mixtures comprising diluents and binding agents may be used, such as Avicel® (microcristalline cellulose), Starlac® (lactose monohydrate 85% with maize starch 15%) or, Ludipress® (lactose monohydrate 93% with Povidone 7%). In a particular embodiment, a 30 mg ulipristal acetate tablet may be manufactured as follows. Lactose monohydrate 79 wt %, ulipristal acetate 10 wt % and povidone 5 wt % are mixed and purified water is added. This granulation step is followed by a drying step in an oven at 40° C. Croscarmellose sodium 5 wt % and magnesium stearate 1 wt % are added for the lubrication step. The obtained formulation is compressed to get the tablet, which shows the following formulation (Table 1).

TABLE 1
30 mg ulipristate acetate tablet:

| | Quantity for one | Quantity for one |
|---|---|---|
| Ingredients | tablet (mg) | tablet (wt %) |
| Ulipristal acetate | 30.00 | 10 |
| Lactose Monohydrate | 237.00 | 79 |
| Povidone | 15.00 | 5 |
| Croscarmellose sodium | 15.00 | 5 |
| Magnesium stearate | 3.00 | 1 |
| Total | 300.00 | 100 |

Further ulipristal acetate tablets are provided hereafter.

TABLE 2
Other ulipristal acetate tablet formulations:

| | 10 mg tablet | 30 mg tablet |
|---|---|---|
| | Quantity for one | Quantity for one |
| Ingredients | tablet in mg (wt %) | tablet in mg (wt %) |
| Ulipristal acetate | 10.00 (10) | 30.00 (10) |
| Lactose Monohydrate | 79.00 (79) | 246.00 (82) |
| Povidone | 5.00 (5) | 9.00 (3) |
| Croscarmellose sodium | 5.00 (5) | 12.00 (4) |
| Magnesium stearate | 1.00 (1) | 3.00 (1) |
| Total | 100.00 (100) | 300.00 (100) |

The subject is provided with a kit comprising i) a dosage form, preferably an oral dosage form such as a tablet, comprising ulipristal acetate and ii) a printed matter stating that ulipristal acetate may be taken within about 120 hours or about 5 days after unprotected intercourse.

Preferably the dosage form comprises about 30 mg ulipristal acetate.

Such printed matter serves as a labelling for the medicine. For instance it is conveniently a leaflet inserted into the packaging of the medicine, or it may be the packaging itself, on which the information is printed.

The FIGURE and examples illustrate the invention without limiting its scope.

### EXAMPLES

### Example 1

### Prospective Multicenter, Open Label Designed Study to Evaluate the Efficacy of Ulipristal Acetate as Emergency Contraception in Women Presenting 48-120 Hours after Unprotected Intercourse (UPI)

### Methods

Women 18 and older who presented for emergency contraception at family planning clinics located in the United States, 48-120 hours after UPI and who met the inclusion/exclusion criteria were enrolled into the study after signing the IRB approved informed consent. The inclusion criteria were regular menstrual cycles 24-35 (+/−5) days in length, no current use of hormonal contraception, willingness not to use hormonal contraception until study completion, and agreement to use barrier methods of contraception from enrollment to study completion. Exclusion criteria included pregnancy, breastfeeding, IUD, tubal ligation or partner vasectomy, and uncertainty about recent menstrual history.

A total of up to three visits were scheduled over the course of the study. The first visit, considered Day 1, included the screening and treatment phases. A high sensitivity urine pregnancy test (level of detection 20 mIU/ml) was performed and a blood sample was taken and stored for later serum quantitative β-hCG testing to exclude pre-existing pregnancy if a pregnancy was detected during the study.

Women were provided daily diaries in which to record further acts of intercourse, contraception used, vaginal spotting or bleeding, concomitant medication and adverse events during study duration.

At follow-up visit (5-7 days after expected onset of menses) a high-sensitivity urine pregnancy test was systematically performed. If the urine pregnancy was positive, this was confirmed by a serum β-hCG test. The pre-treatment serum specimen was also assayed for β-hCG to verify whether the pregnancy pre-dated intake of the study drug. Confirmed pregnancies were further evaluated by serum quantitative hCG(s) and transvaginal ultrasound(s) to determine the estimated fertilization date.

Women could enroll in the study more than once but they must have completed prior study participation before re-enrolling.

The primary efficacy measurement was the pregnancy rate, defined as the number of pregnancies after administration of ulipristal acetate for EC divided by the number of women treated. The primary efficacy analysis compared this pregnancy rate to the pregnancy rate that would have been expected in the absence of EC treatment, which was calculated according to Trussell's method (Trussell et al, 1998) using the pooled recognizable set of conception probabilities and the estimated cycle day of UPI based on self-reported date of last menstrual period, cycle length and date of UPI. The observed pregnancy rate was considered to be statistically significantly lower than the expected pregnancy rate if the upper bound of the 2-sided 95% confidence interval of the observed pregnancy rate calculated using the Agresti-Coull method (A. Coull, 1998) was below the estimated expected pregnancy rate.

The main secondary efficacy analysis compared the upper bound of the 95% confidence interval of the observed pregnancy rate to a clinical irrelevance threshold of 4%. This threshold corresponds to a reduction by half of the expected 8% pregnancy rate in the absence of contraception as observed in previous international studies (Von Hertzen et al, 1998; Piaggio, 1999; Von Hertzen et al, 2002). The study was to be considered a success only if both the primary efficacy and the main secondary analyses were conclusive. Other secondary analyses included calculation of the prevented fraction, defined as the number of pregnancies prevented (expected minus observed) divided by the number of pregnancies expected, and analysis of trend in pregnancy rates over time (by 24-hour interval) using a logistic regression model.

The population analyzed for primary efficacy excluded women who were lost to follow-up and women ages 36 and older due to reduced fertility in this age category based upon FDA guidance. Further participations in the study allowed by protocol were also excluded from primary efficacy evaluation, as well as pregnancies that were determined as not compatible with study drug failure by a Data Safety Monitoring Board (DSMB) consisting of independent experts.

The sample size was estimated in order to reach at least 80% power for statistical analyses comparing the observed pregnancy rate to the expected pregnancy rate as well as to a clinical irrelevance threshold (set at 4%, corresponding to a 50% reduction in the expected pregnancy rate as estimated in previous clinical trials of EC methods (11;12) (von Hertzen et al 1998; von Hertzen et al 2002). Using the hypothesis of a 2.5% pregnancy rate with ulipristal acetate for treatment 48-120 h after UPI, 1200 subjects were needed to demonstrate that the pregnancy rate was lower than a clinical irrelevance threshold of 4%. Study enrolment was stopped once 1200 subjects meeting the criteria for the primary efficacy analysis had completed the study.

### Results

Overall 1623 requests for EC led to screening for enrolment into the study, of which 90 were screen failures, leaving a number of women treated of 1533 which comprised 1449 unique women plus 84 EC treatments upon subsequent (repeat) enrollment. Demographics and baseline characteristics as well as safety information are described for all women treated (Safety Population). Pregnancy status was unknown in 106 women (6.9%) mainly because they were lost to follow-up. Of the Safety population, 1241 were eligible for inclusion in population used for primary efficacy analysis (Primary Efficacy Population); those excluded were for reasons of age greater than 35 (99 women), unknown pregnancy status at follow-up (106 women), repeat enrollment (84 women) and pregnancies deemed not compatible with EC failure by DSMB (3 women).

### Baseline Characteristics

Demographic and baseline characteristics were similar between the Safety Population and the Primary Efficacy population (data not shown).

Data from the obstetric and gynecological history showed that more than one-half of women reported a history of pregnancy (52.4%), and a similar proportion past EC use (52.5%). Women enrolled reported a mean menstrual cycle length at screening of 29 days and requested EC primarily for reasons of intercourse without contraception (72.3%) or condom breakage or slippage (25.3%). Just over half of the participants (55.6%) took the study drug 48-72 hours after UPI, with the others presenting later.

Women reported UPI throughout the entire cycle (range: cycle day 1 to 41). UPI tended to have taken place during the peri-ovulatory fertile window. Indeed, the majority of women (52.5%) presented with UPI that took place between cycle days 10 and 20.

### Efficacy

A total of 29 pregnancies were detected at follow-up in women enrolled in this study. Three pregnancies were excluded from the primary efficacy analysis population as the DSMB determined that they were not compatible with emergency contraception failure (i.e. 1 pre-treatment and 2 post-treatment pregnancies). The Primary Efficacy Population comprised 1241 women with 26 pregnancies, for an overall pregnancy rate of 2.1% (95% CI; 1.4%, 3.1%). The expected pregnancy rate using Trussell's methodology was 5.5%, meaning that 69 pregnancies would have been expected in the Primary Efficacy Population had no EC been given (Trussell et al, 1998). The upper limit of the 2-sided 95% CI of the observed pregnancy rate (3.1%) was therefore significantly lower than the expected pregnancy rate as well as the clinical irrelevance threshold (4%), so the results met the protocol definition of study success. Efficacy was also confirmed in the Safety Population of 1533 women where the observed pregnancy rate was 1.9% (95% CI; 1.3%, 2.8%) compared to an expected pregnancy rate of 5.7%. The proportion of pregnancies prevented by ulipristal acetate overall was 62.3% (95% CI: 41.9%-75.6%). The pregnancy rates and prevented pregnancy fraction were analyzed by 24-hour time intervals after UPI, as presented in Table 3 below.

TABLE 3
Efficacy according to time after UPI

| mITT population | All | 48-72 h | >72-96 h | >96-120 h |
|---|---|---|---|---|
| Exposed (n) | 1241 | 693 | 390 | 158 |
| Expected Pregnancies (n)* | 69 | 42 | 19 | 8 |
| Observed Pregnancies (n) | 26 | 16 | 8 | 2 |
| Expected Pregnancy Rate (%) | 5.5% | 6.0% | 5.0% | 4.9% |
| Observed Pregnancy Rate (%) | 2.1% | 2.3% | 2.1% | 1.3% |
| [95% CI] | [1.4%-3.1%] | [1.4%-3.8%] | [1.0%-4.1%] | [0.1%-4.8%] |
| Effectiveness (Prevented Fraction) | 62.3% | 61.9% | 57.9% | 75.0% |
| [95% CI] | [41.9%-75.6%] | [36.3%-77.2%] | [14.6%-79.2%] | [6.2%-93.3%] |

*Based on pooled recognizable set of conception probabilities (Trussell et al, 1998)

There is no evidence of change in efficacy over time as confirmed when testing the linear trend of pregnancy rates over time in a logistic regression model (p=0.2490). It should be noted that 14 of the 26 observed pregnancies occurred in women whose UPI took place outside of the presumed fertile window that extends from day −5 to day +1 relative to ovulation.

### Discussion

This study demonstrates that a single 30 mg dose of ulipristal acetate is effective when used as emergency contraception 48-120 hours following UPI. Of particular clinical relevance is the sustained efficacy of ulipristal acetate up to 120 hours of unprotected intercourse.

The currently available EC drug levonorgestrel is approved for use up to 72 hours following unprotected intercourse and is also used off-label beyond 72 hours, but its efficacy has been shown to decrease in a statistically significant fashion over time (von Hertzen et al, 1998; von Hertzen et al, 2002). The time-dependent nature of levonorgestrel's efficacy was one of the main driving factors in widespread efforts to render EC easily and rapidly accessible for women in need, including making levonorgestrel EC available on an over-the-counter basis in some 30 countries worldwide. Despite broad scale educational campaigns regarding the importance of early EC intake for optimal efficacy, significant numbers of women continue to present several days after unprotected intercourse. According to data from a large WHO study of EC (Von Hertzen et al, 2002), one in 10 women present more than 72 hours following unprotected intercourse. As ulipristal acetate has already been shown to be as effective as levonorgestrel for intake 0-72 hours following unprotected intercourse (Creinin et al, 2006), this study was designed to obtain evidence regarding efficacy in the late intake time window, hence the enrolment starting at 48 hours and later. Indeed, almost 50% of women in this study presented for EC more than 72 hours after unprotected intercourse. Insertion of a copper intra-uterine device (IUD) is a highly effective but poorly accessible method of EC. There is therefore a clear need for a highly effective hormonal method of EC for late intake.

This study was designed in compliance with international regulatory agencies including the FDA, and its results are strengthened by the study's prospective design and large, geographically and racially/ethnically diverse cohort. Additionally, the proportion of women that were lost to follow-up after study drug intake was small, though the limited follow-up regarding continuing pregnancy outcomes is a study limitation.

In conclusion, the results of this study demonstrate that ulipristal acetate prevents pregnancies when used as EC up to 120 hours after intercourse, making it the first hormonal method of EC with solid evidence of efficacy for late intake.

### Example 2

### Prospective Multicenter, Single-Blind Designed Study to Evaluate the Efficacy of Ulipristal Acetate as Emergency Contraception (EC) in Women Presenting 0-120 Hours after Unprotected Intercourse (UPI), in Comparison with Levonorgestrel

### Methods

This was a prospective, single-blind (subject and sponsor blind, investigator not blind), randomized, multicenter, 2-arm parallel comparative study designed to evaluate the efficacy of a single dose of ulipristal acetate (30 mg tablets) compared to levonorgestrel (1.5 mg) administered for EC within 120 hours after unprotected intercourse. It was performed in 10 centers in Europe and 25 centers in the US. Women (aged ≧16), with regular menstrual cycles (between 24 and 35 days and intra-individual variations less than or equal to 5 days), who presented for EC within 120 hours after unprotected intercourse at a participating study site and who met the inclusion/exclusion criteria were enrolled into the study after they signed informed consent form. Subjects were excluded from the study in case of ongoing pregnancy or breast-feeding or current use of hormonal contraception or IUD.

The study medication (ulipristal acetate 30 mg or levonorgestrel 1.5 mg) was administered according to a random allocation procedure generated electronically. Treatment was administered orally immediately after all eligibility criteria (including negative urine pregnancy test) had been verified. At follow-up visit (5-7 days after expected onset of menses) a high-sensitivity urine pregnancy test was systematically performed. If the urine pregnancy was positive, this was confirmed by a serum β-hCG test. The pre-treatment serum specimen was also assayed for β-hCG to verify whether the pregnancy pre-dated intake of the study drug. Confirmed pregnancies were further evaluated by serum quantitative hCG(s) and transvaginal ultrasound(s) to determine the estimated fertilization date.

Based on previous similar clinical trials, pregnancy rates in the ulipristal acetate and levonorgestrel group were expected to be 1% and 1.7% respectively. In order to demonstrate the non-inferiority of ulipristal acetate to levonorgestrel for patients within 72 hours of unprotected intercourse, 827 patients per treatment group were needed. For this comparison, the non-inferiority margin was set to 1.6 in odds ratio (a non-inferiority margin of 1.6 in odds ratio is equivalent to a non-inferiority margin of 1% in percent point with an assumed pregnancy rate with levonorgestrel of 1.7%) with a type I error rate of 5% (two-sided) and 85% power. In order to compensate for an anticipated lost to follow-up rate of 10%, 910 patients per group had to be included in the 0-72 hour time interval. Furthermore, recruitment of patients requesting emergency contraception between 72 h and 120 h was estimated to represent 1 out of 10 patients. Therefore, taking into account recruitment in the 72-120 h interval, 1022 patients per group were to be randomized for a total of 2044 patients.

The efficacy analysis was performed on the modified Intent To Treat (mITT) population which included all subjects who had received study drug, were participating in the study for the first time (multiple enrolments were allowed in the protocol), had a known pregnancy status after emergency contraception intake, were aged up to and including 35 years, and did not have a pregnancy identified as having started before ulipristal acetate intake or not compatible with study drug failure, based on independent evaluation.

In order to ensure unbiased evaluation of pregnancy data, an independent, autonomous DSMB composed of two experts in the field of gynecology, one methodologist and one expert in ethical questions was established to review incidence of pregnancy with respect to unacceptability threshold and give recommendations during the course of the clinical trial. In addition, the DSMB assessed whether each pregnancy was “compatible” or “not compatible” with treatment failure based on available data.

### Results

### Subject Disposition

Two thousand three hundred twenty one (2321) subjects were screened, signed informed consent and were enrolled into the study. Among the 2321 screened subjects, 100 were not treated mainly for not compliance to inclusion/exclusion criteria. One thousand one hundred four (1104) subjects were treated with ulipristal acetate; 1117 subjects received levonorgestrel. Among the 1104 ulipristal acetate treated subjects, 1013 (91.8%) completed all scheduled study visits. Of the 91 ulipristal acetate treated subjects who discontinued the study, 48 were lost to follow-up (corresponding to 4.3% of treated subjects), 36 were discontinued for other reasons and 5 withdrew consent. Among the 1117 levonorgestrel treated subjects, 1046 (93.6%) completed all scheduled study visits. Of the 71 treated subjects who discontinued the study, 40 were lost to follow-up (corresponding to 3.6% of treated subjects), 30 were discontinued for other reasons, and 1 withdrew consent. No subject was discontinued due to an AE.

Overall 1694 mITT subjects (ulipristal acetate, 851; levonorgestrel, 843) were analyzed within the 72 hour time window and 1893 were analyzed in the 120 hour time window. Six patients were enrolled 120 hours and more after unprotected intercourse and were not included in mITT analyses. However none of them become pregnant.

### Demography and Baseline Characteristics

Subject demographics in the ITT population were similar for both treatment groups in age (mean 24.5 and 24.9 years old). The distribution of race was similar with the majority White (72.4 and 72.8%) or Black or African American (18.5 and 19.0%). The two groups were evenly matched with respect to height and weight (mean BMI 25.2 and 25.3). The results are similar to those observed in all other study populations.

The average menstrual cycle length at screening was 28.7 days (ulipristal acetate, 28.7 days; levonorgestrel, 28.8 days) with a range of 23-40 days (ulipristal acetate, 24-35 days; levonorgestrel, 23-40 days). The majority of subjects (ulipristal acetate, 98.6%; levonorgestrel, 98.7%) had regular periods in the previous year with an average of 4.7 bleeding days (ulipristal acetate, 4.7 days; levonorgestrel, 4.7 days). Very few subjects (ulipristal acetate, 0.8%; levonorgestrel, 1.3%) reported intermenstrual bleeding in the past three months at inclusion and 2.6% (ulipristal acetate, 2.5%; levonorgestrel, 2.7%) had a history of amenorrhea or oligomenorrhea. Previous pregnancies were reported by 47.5% (ulipristal acetate, 47.3%; levonorgestrel, 47.8%) of the subjects. Male condom use was the primary contraceptive method declared in the past three months at inclusion (ulipristal acetate, 82.1%; levonorgestrel, 83.7%). Fifty five percent (55.3%) of subjects (ulipristal acetate, 54.9%; levonorgestrel, 55.7%) had used EC prior to study entry. The gynecological history results are comparable between treatment groups.

Eight hundred five (36.2%) ITT subjects (ulipristal acetate, 37.0%; levonorgestrel, 35.5%) reported having had protected intercourse more than 120 hours before EC intake, only 4 of the subjects (ulipristal acetate, 1; levonorgestrel, 3) reported unprotected intercourse. The distribution of subjects according to time from unprotected intercourse to treatment intake was similar between the two treatment groups. The majority of unprotected intercourse occurred between Day 10 to Day 21 in both treatment groups. One thousand four hundred twenty six (64.2%) ITT subjects (ulipristal acetate, 65.2%; levonorgestrel, 63.2%) had further intercourse after study medication; the majority of these subjects (overall, 91.7%; ulipristal acetate, 91.9%; levonorgestrel, 91.4%) had exclusively protected intercourse. Of the 119 subjects with further unprotected intercourse, 43 had unprotected intercourse that led to further EC intake.

### Efficacy Results

For ulipristal acetate subjects in the entire study population (n=843) treated within 72 hours of unprotected intercourse, the observed pregnancy rate of 1.78% (95% CI; 1.04%, 2.98%) was statistically significantly lower than the expected pregnancy rate of 5.54%. For levonorgestrel treated subjects (n=851), the observed pregnancy rate of 2.59% (95% CI; 1.68%, 3.94%) was also statistically significantly lower than the expected pregnancy rate of 5.43%. Pregnancy rates were also analyzed for the mITT subjects who had unprotected intercourse within 120 hours of EC intake. Of 939 ulipristal acetate treated subjects, the observed pregnancy rate 1.60% (95% CI, 0.93%, 2.67%) was statistically significantly lower than the expected pregnancy rates of 5.72%.

The upper bound of the 2-sided 95% CI of the observed pregnancy rate for both the mITT populations within 72 hours and within 120 hours of ulipristal acetate treatment intake is below the clinical irrelevance threshold (4%).

Table 4 summarizes the efficacy results obtained in this study.

TABLE 4
Pregnancy rates for treatment within 72 hours
or 120 hours of unprotected intercourse

Pregnancy Rate n (%)

| | Ulipristal acetate | Levonorgestrel |
|---|---|---|
| 0-72 h mITT | 15 (1.8) | 22 (2.6) |
| (n = 1694) | | |
| 72-120 h mITT | 0 (0.0) | 3 (2.8) |
| (n = 203) | | |
| 0-120 h mITT | 15 (1.6) | 25 (2.6) |
| (n = 1899) | | |

Noteworthy, no pregnancy was observed with ulipristal acetate administered from 72 to 120 hour after UPI.

See also the FIGURE.

The observed pregnancy rates at the five 24-hour time intervals between 0 to 120 hours from unprotected intercourse to treatment are summarized in Table 5. In the mITT population the observed pregnancy rates for the ulipristal acetate group were 1.60%, 2.13% and 1.48%, respectively at 0-24, >24-48 and >48 to 72 hour intervals. No pregnancies were observed at the >72 to 96 and >96 to 120 hour intervals.

TABLE 5
Observed pregnancy rates at 24-hr time intervals
from unprotected intercourse to treatment (mITT
population, ulipristal acetate treatment group)

| Time intervals | N of | Pregnancy | Odds | Odds ratios |
|---|---|---|---|---|
| (hours) | subjects | Rate (%) | ratio | 95% CI* |
| >0 to <=24 | 312 | 1.60 | NA* | NA* |
| >24 to <=48 | 329 | 2.13 | 1.33 | [0.42-4.25] |
| >48 to <=72 | 203 | 1.48 | 0.69 | [0.18-2.70] |
| >72 to <=96 | 63 | 0.0 | 0.00 | 0.0->999 |
| >96 to <=120 | 34 | 0.0 | NA* | NA |

*NA, not applicable
**Odd ratio at a given time interval is relative to the previous 24 hrs time interval

The odds ratios (compared with that of the previous 24 hour interval) were 1.33 (95% CI; 0.42%, 4.25%) and 0.69 (95% CI; 0.18%, 2.70%) at the >24 to 48 and >48 to 72 hour intervals from unprotected intercourse to EC intake. The odds ratios for the levonorgestrel treatment group at the five 24-hour time intervals (compared with that of the previous 24 hour interval) were 0.73, 1.17, 1.08 and 1.11, respectively.

In the mITT population, treatment with ulipristal acetate prevented 68.1% (95% CI; 45.80%, 81.21%) of expected pregnancies and treatment with levonorgestrel prevented 52.2% (95% CI; 25.12%, 69.45%) of expected pregnancies when unprotected intercourse was within 72 hours of EC intake (p=0.253). If unprotected intercourse was within 120 hours of EC intake, ulipristal acetate treatment prevented 72.2% (95% CI; 52.78%, 83.66%) of expected pregnancies and levonorgestrel treatment prevented 52.8% (95% CI; 27.80%, 69.18%) of expected pregnancies (p=0.127). Compared to the expected pregnancy rate, significantly more pregnancies were prevented with ulipristal acetate than levonorgestrel (p<0.05 for a one-sided randomization test performed on mITT population treated within 120 hours of unprotected intercourse and p<0.05 for a two-sided randomization test performed on mITT population treated between 72 and 120 hours after unprotected intercourse).

### Discussion

This large multicenter international study demonstrated that a single 30 mg dose of ulipristal acetate as emergency contraception whether administered within 72 or 120 hours after unprotected intercourse, statistically significantly lowered the observed pregnancy rate compared to the expected pregnancy rate in the absence of EC. The reduction in pregnancy rate was clinically relevant. The results were robust and conclusive and meet the protocol definition of study success. The efficacy of ulipristal acetate was also supported by an analysis of the trend in pregnancy rates up to 120 hours based upon estimates of the probability of pregnancy from the time of unprotected intercourse to treatment assessed at various time intervals. These analyses show a sustained effect of ulipristal acetate on pregnancy prevention up to 120 hours.

This study demonstrates that ulipristal acetate is an effective EC with no apparent loss of effectiveness if taken up to 120 hours after unprotected intercourse. This represents a clinically important and significant improvement with respect to currently available methods.

### Example 3

### Pooled Efficacy Analysis

When the efficacy data of women treated with ulipristal acetate in the two trials (Example 1 and Example 2) are grouped in a pooled analysis, further evidence of the efficacy of the method is provided. In particular, day-by-day the observed pregnancy rate is statistically significantly lower than the pregnancy rate expected in the absence of treatment, demonstrating that ulipristal is highly effective over each individual 24-h period (see Table 6).

TABLE 6
Pooled efficacy analysis

| mITT pooled population | 0-24 h | >24-48 h | >48-72 h | >72-96 h | >96-120 h |
|---|---|---|---|---|---|
| Women treated total (Example | 313 | 338 | 881 | 455 | 193 |
| 1 and 2) | | | | | |
| Observed pregnancy rate (%) | — | — | 2.3% | 2.1% | 1.3% |
| (Example 1) | | | | | |
| Observed pregnancy rate (%) | 1.6% | 2.1% | 1.5% | 0% | 0% |
| (Example 2) | | | | | |
| Overall observed pregnancy | 1.60% | 2.07% | 2.16% | 1.76% | 1.04% |
| rate (%) (pooled) | (0.57-3.79) | (0.92-4.30) | (1.36-3.37) | (0.83-3.49) | (0.04-3.94) |
| [95% CI] | | | | | |
| Overall expected pregnancy | 4.83% | 5.88% | 6.02% | 5.26% | 5.49% |
| rate (%) (pooled) (Trussell's | | | | | |
| method) | | | | | |

### REFERENCES

A. Coull B A. Approximate is better than “exact” for interval estimation of binomial Proportion. Amer. Statist. 1998; 52:119-126;
Creinin M D, Schlaff W, Archer D F, Wan L, Frezieres R, Thomas M, Rosenberg M, Higgins. Progesterone Receptor Modulator for Emergency Contraception: A Randomized Controlled Trial. Obstetrics & Gynecology 2006; Vol. 108; No. 5: 1089-97;
Croxatto H B, Brache V, Pavez M, Cochon L, Forcelledo M L, Alvarez F, Massai R, Faundes A, Salvatierra A M. Pituitary-ovarian function following the standard levonorgestrel emergency contraceptive dose or a single 0.75-mg dose given on the days preceding ovulation. Contraception 2004; 70: 442-450;
Grimes D. Intrauterine Devices. In Hatcher Ra et al, ed. Contraceptive Technology. New York: Ardent Media, 18th ed, 2004, p. 499-502;
Marions L, Cekan S Z, Bygdeman M, Gemzell-Danielsson K. Effect of emergency contraception with levonorgestrel or mifepristone on ovarian function. Contraception 2004; Vol 69: 373-7;
Stratton P, Hartog B, Hajizadeh N, Piquion J, Sutherland D, Merino M, Lee Y J, Nieman L K. A single mid-follicular dose of CDB-2914, a new antiprogestin, inhibits folliculogenesis and endometrial differentiation in normally cycling women. Hum Reproduction 2000 May; 15(5):1092-9;
Piaggio G, von Hertzen H, Grimes D A, Van Look P F. Timing of emergency contraception with levonorgestrel or the Yuzpe regimen. Task Force on Postovulatory Methods of Fertility Regulation. Lancet 1999; 353 (9154): 697-702;
Trussell J, Rodriguez G, Ellertson C. New estimates of the effectiveness of the Yuzpe regimen of emergency contraception. Contraception. 1998; 57(6): 363-9;
Von Hertzen H, Piaggio G, Van Look P F. Emergency contraception with levonorgestrel or the Yuzpe regimen. Task Force on Postovulatory Methods of Fertility Regulation. Lancet 1998; 352(9144): 428-33;
Von Hertzen H, Piaggio G, Ding J, Chen J, Song S, Bartfai G. Low dose mifepristone and two regimens of levonorgestrel for emergency contraception: a WHO multicentre randomised trial. Lancet 2002; 360 (9348): 1803-10;
Wilcox A J, Weinberg C R, Baird D D. Timing of Sexual Intercourse in Relation to Ovulation. NEJM 1995; Vol. 33: 1518-21;
Wilcox A J, Baird D D, Dunson D B, McConnaughey D R, Kesner J S, Weinberg C R. On the frequency of intercourse around ovulation: evidence for biological influences. Human Reproduction 2004: Vol. 19: 1539-43;

1. A method for providing post coital contraception in a female subject, comprising providing the subject with a therapeutically effective amount of ulipristal acetate, between about 3 to about 5 days after unprotected intercourse.

2. The method of claim 1 wherein post coital contraception is provided more than about 72 hours after unprotected intercourse.

3. The method of claim 2, wherein post coital contraception is provided more than about 96 hours after unprotected intercourse.

4. The method of claim 3, wherein post coital contraception is provided about 120 hours after unprotected intercourse.

5. The method of claim 1, wherein the post coital contraception is an emergency contraception.

6. The method of claim 1, wherein the ulipristal acetate is administered in an oral dosage form.

7. The method of claim 6 wherein the oral dosage form is a tablet.

8. The method of claim 1, wherein the dosage form comprises about 30 mg ulipristal acetate.

9. The method of claim 1, wherein the ulipristal acetate is administered in a form suitable for buccal, parenteral, transdermal, vaginal, or uterine route.

10. A kit comprising i) a dosage form comprising ulipristal acetate and ii) a printed matter stating that ulipristal acetate may be taken within about 120 hours or about 5 days after unprotected intercourse.

11. The kit of claim 10, wherein the dosage form is an oral dosage form.

12. The kit of claim 11, wherein the oral dosage form is a tablet.

13. The kit of claim 11, wherein the oral dosage form comprises about 30 mg ulipristal acetate.
